(19) United States
(12) Patent Application Publication
(10) Pub. No.: US 2011/0228219 A1
(43) Pub. Date: Sep. 22, 2011

(54) OPHTHALMIC SIMULATOR

(75) Inventors: Hwa-Soo Lee, Tokyo (JP); Takazo Yamada, Tokyo (JP); Kyoko Fujita, Tokyo (JP)

(73) Assignee: NIHON UNIVERSITY, Chiyoda-ku (JP)

(21) Appl. No.: 13/131,211

(22) PCT Filed: Nov. 12, 2009
(86) PCT No.: PCT/JP2009/006061

§ 371 Date: May 25, 2011

(30) Foreign Application Priority Data
Nov. 25, 2008 (JP) 2008-300214

(51) Int. Cl. A61B 3/10

(52) U.S. Cl. 351/205

(57) ABSTRACT

A metamorphopsia simulator is capable of numerically simulating deformation of the retina of a metamorphopsia patient and irregularity in an arrangement of visual cells caused thereby. The metamorphopsia simulator of the present invention uses a method of digitizing a deformation amount of the retina according to an expression based on a probability density function, to find a movement amount of visual cells, and according to the movement amount of visual cells, find distortion of an image observed by the metamorphopsia patient.

### BACKGROUND OF THE INVENTION

1. Field of the Invention

The present invention relates to a technique of simulating distortion of an image observed by an eye disease patient according to a deformation amount of the retina of the patient and analyzing the condition of the eye disease patient.

2. Description of Related Art

In an eyeball illustrated in FIG. 1, light passes through a cornea 11 and is focused by a crystalline lens 13 to project an image on a retina 15. The light projected on the retina 15 is caught by a plurality of visual cells 19 existing on the retina 15 and is transmitted through optic nerves 21 to the brain that recognizes the image. The image recognized by the brain is a composition of discrete data caught by the respective visual cells 19 and is considered, from the view point of information processing, to be a result of digital processing conducted by the brain.

On the central nose side of the retina 15, there is an optic papilla that is a bundle of the optic nerves 21 leading to the brain, and around which, there is a part called a macula 17 as illustrated in FIG. 1, The macula 17 has a crater-like concave shape having a width of about 0.5 mm and a depth of about 0.3 mm. At this part on the retina, a concentration of the visual cells is extremely high compared with the rest of the retina, and therefore, the part is considered to be the nerve center of eyesight. Accordingly, if any disease causes abnormality in the macula 17, the shape of a viewed object is distorted. This phenomenon is a symptom of an eye disease called metamorphopsia. Known causes of the disease include an epiretinal membrane, a macular hole, age-related macular degeneration, central serous chorioretinopathy, and the like. The number of patients suffering from the disease is not small.

If abnormality occurs in the macula, taking an eyeground photograph enables the abnormality in the macula shape to be measured quantitatively. Namely, the disease mentioned above is diagnosable. It is difficult, however, to quantitatively determine how the patient suffering from the disease recognizes an image. In medical practice, an Amsler test is carried out.

The Amsler test uses an Amsler chart illustrated in FIG. 2 and is conducted as mentioned below.

The grid-like chart illustrated in FIG. 2 is prepared. The number of grid lines in the chart is 21 in each direction and they are arranged at intervals of 5 mm. At a position of 30 cm from the chart, a patient observes the chart with an eye to be diagnosed and draws on the chart how the chart is viewed. This method is able to determine, if the patient has the disease on only one eye, how the patient perceives the image. The method, however, is unable to allow a doctor to know an image recognized by the patient if both eyes of the patient are suffering from the disease. Even if the patient has abnormality in only one eye, it is hard for the patient to correctly draw an image seen by the patient. Accordingly, the method is able only to qualitatively determine deformation of the image.

There is a related art employing a metamorphopsia test chart including plural dotted lines of predetermined length including a dotted line of narrowest dot intervals, a dotted line of widest dot intervals, and dotted lines of intermediate dot intervals. (Refer to Patent Literature 1.)

Also, there is a metamorphopsia diagnosis chart involving plural straight lines that form grids to diagnose metamorphopsia. The diagnosis chart has, from a central fixation target 2 and within a visual angle of 20 degrees in vertical and horizontal directions, vertical and horizontal straight lines at visual angle intervals of one degree, to form square grids. If the retina of a patient is deformed due to an influence of, for example, edema, an image projected on the retina distorts, i.e., the patient perceives distorted straight lines. The patient observes the diagnosis chart 1 in a perpendicular direction in front of the fixation target 2, to test whether there is any location where the straight lines are distorted and examine the degree of distortion and the like. (Refer to Patent Literature 2.)

These, however, only use metamorphopsia diagnosis charts to simply test the degree of metamorphopsia.

Deformation of an image viewed by a metamorphopsia patient occurs at the macula as mentioned above, i.e., at the center of a view field of the patient. If a doctor can grasp the degree of image deformation due to the disease, the doctor will be able to care for not only the eye disease but also mental anxiety caused by the disease. It is very important in treating metamorphopsia that a doctor quantitatively acquires an image recognized by a patient.

Patent Literature 1: Japanese Unexamined Patent Application Publication No. 2001-149314

Patent Literature 2: Japanese Unexamined Patent Application Publication No. 2003-265412

### SUMMARY OF THE INVENTION

A problem in the ophthalmic simulator is that it is unable to numerically simulate deformation of the retina of an eye disease patient or irregularity to be caused thereby in an arrangement of optic nerves.

An ophthalmic simulator according to the present invention is characterized in that it includes a retina deformation analyzing unit to mathematically analyze, based on a shape of a retina after deformation, a deformation amount with respect to a shape of the retina before deformation and a visual cell movement analyzing unit to mathematically analyze, based on the deformation amount of the retina analyzed by the retina deformation analyzing unit, a movement amount of visual cells due to the deformation of the retina.

The ophthalmic simulator according to the present invention may have a metamorphopsic image reproducing unit to reproduce, based on the movement amount of visual cells analyzed by the visual cell movement analyzing unit, distortion of an image observed by the eye disease patient.

The retina deformation analyzing unit of the ophthalmic simulator according to the present invention may approximate the deformation amount of the retina of the eye disease patient according to the below-mentioned Expression 1 based on a probability density function with a visual line being in a vertical direction of the retina, a direction orthogonal to the visual line being a horizontal direction of the retina, a movement amount in the vertical direction of the retina being Z, a maximum value of deformation of the retina being A, a distance in the horizontal direction from an origin being x, and a standard deviation in the probability density function being σ:

$$Z = A \cdot exp \frac{- {x}^{2}}{2 {σ}^{2}} \qquad ( Expression 1 )$$

The ophthalmic simulator according to the present invention includes the retina deformation analyzing unit to mathematically analyze, based on a shape of a retina after deformation, a deformation amount with respect to a shape of the retina before deformation and the visual cell movement analyzing unit to mathematically analyze, based on the deformation amount of the retina analyzed by the retina deformation analyzing unit, a movement amount of visual cells due to the deformation of the retina. Accordingly, the simulator is capable of numerically simulating deformation of the retina of an eye disease patient and irregularity to be caused thereby in an arrangement of optic nerves of the patient.

The ophthalmic simulator according to the present invention has the metamorphopsic image reproducing unit to reproduce, based on the movement amount of visual cells analyzed by the visual cell movement analyzing unit, distortion of an image observed by the eye disease patient, to allow the distortion of an image observed by the eye disease patient to be examined.

The retina deformation analyzing unit of the ophthalmic simulator according to the present invention approximates a deformation amount of the retina of the eye disease patient according to the below-mentioned Expression 1 based on a probability density function with a visual line being in a vertical direction of the retina, a direction orthogonal to the visual line being a horizontal direction of the retina, a movement amount in the vertical direction of the retina being Z, a maximum value of deformation of the retina being A, a distance in the horizontal direction from an origin being x, and a standard deviation in the probability density function being σ:

$$Z = A \cdot exp \frac{- {x}^{2}}{2 {σ}^{2}} \qquad ( Expression 1 )$$

Accordingly, it is possible to simulate a deformation amount of the retina of the eye disease patient according to the expression based on a probability density function and numerically express the deformation amount of the retina.

### BRIEF DESCRIPTION OF DRAWINGS

FIG. 1 shows a schematic view of an eyeball;

FIG. 2 shows an example of an Amsler chart;

FIG. 3 shows a functional block diagram of a metamorphopsia simulator according to an embodiment of the present invention;

FIG. 4 shows an example of a shape to recognize;

FIG. 5 shows an arrangement of visual cells on the retina;

FIG. 6 shows data about the visual cells;

FIG. 7 shows an example of irregularity in the arrangement of visual cells;

FIG. 8 shows data obtained from the visual cells of the irregular arrangement;

FIG. 9 shows a shape reprocessed without considering the irregular arrangement;

FIG. 10 shows a flowchart of simulation;

FIG. 11 shows a model of retina deformation around the macula;

FIG. 12 shows a schematic view of positions of visual cells before and after deformation of the retina;

FIG. 13 shows a coordinate system for simulation;

FIGS. 14(A)-14(E) show examples of calculated stepped shapes of the retina;

FIGS. 15(A)-15(E) show movement amounts of visual cells in a horizontal direction;

FIGS. 16(A)-16(C) show metamorphopsic images prepared by simulation; and

FIGS. 17(A) and 17(B) show recognized images of a character prepared by simulation.

### DETAILED DESCRIPTION OF EMBODIMENTS

The problem of the ophthalmic simulator that deformation of the retina of an eye disease patient and irregularity to be caused thereby in an arrangement of visual cells are unable to numerically simulate is solved by simulating and analyzing a deformation amount of the retina of the eye disease patient and a movement amount of visual cells before and after the deformation according to a mathematical expression.

### EMBODIMENTS

A metamorphopsia simulator as an embodiment of the ophthalmic simulator according to the present invention will be explained.

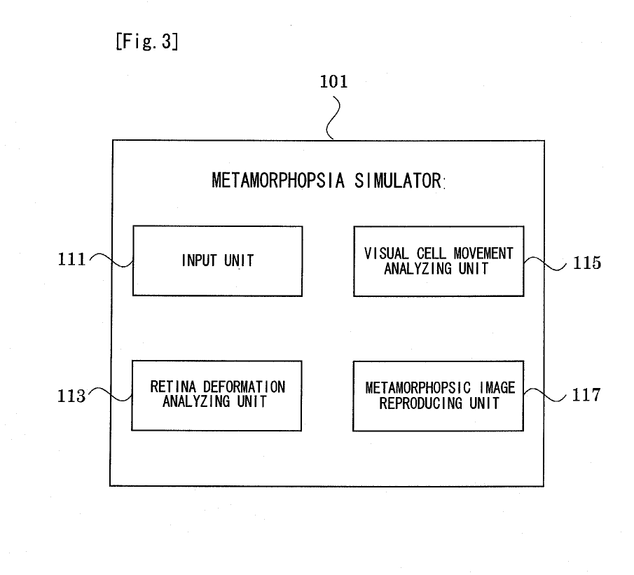

FIG. 3 is a functional block diagram illustrating the metamorphopsia simulator 101 according to an embodiment of the present invention.

The metamorphopsia simulator 101 has function blocks including an input unit 111, a retina deformation analyzing unit 113, a visual cell movement analyzing unit 115, and a metamorphopsic image reproducing unit 117.

The input unit 111 receives an image of a test chart observed by a metamorphopsia patient (eye disease patient). An example of a difference between an original image of the test chart and an image thereof observed by the metamorphopsia patient will be explained later in detail.

The retina deformation analyzing unit 113 analyzes, based on an image obtained by, for example, photographing the eyeground of the metamorphopsia patient, a deformed shape of the retina of the patient and approximates a deformation amount with respect to a shape of the retina before deformation according to an expression (Expression 1 to be explained later in detail) based on a probability density function.

The retina deformation analyzing unit 113 also has a function of analyzing a deformation amount of the retina according to a deviation from a normal position, i.e., a movement amount of visual cells due to the deformation of the retina of the metamorphopsia patient obtained by the visual cell movement analyzing unit 115.

The visual cell movement analyzing unit 115 analyzes the deviation of visual cells of the metamorphopsia patient from the normal position according to the deformation amount of the retina analyzed by the retina deformation analyzing unit 113.

The visual cell movement analyzing unit 115 also has a function of analyzing the deviation (movement amount) of visual cells from the normal position according to the image of the test chart observed by the metamorphopsia patient received through the input unit.

The metamorphopsic image reproducing unit 117 reproduces and outputs distortion of the image observed by the metamorphopsia patient according to the deviation (movement amount) of visual cells of the metamorphopsia patient from the normal position analyzed by the visual cell movement analyzing unit 115.

The metamorphopsia simulator 101 has a function of finding the deviation of visual cells from the normal position according to a deformation amount of the retina of the metamorphopsia patient, and according to the deviation, reproducing deformation of the image observed by the metamorphopsia patient. Also, as a reverse process, the metamorphopsia simulator 101 has a function of finding the deviation of visual cells from the normal position according to distortion of the image of the test chart observed by the metamorphopsia patient, and according to the deviation, finding a deformation amount of the retina.

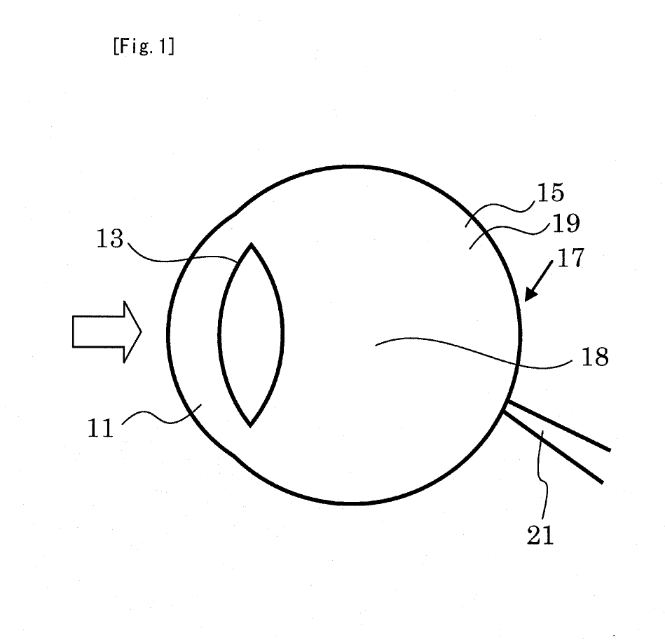

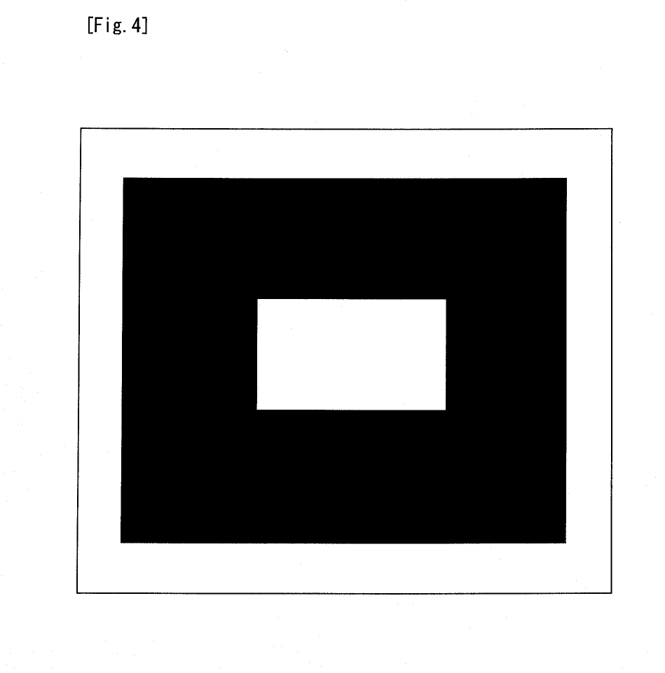

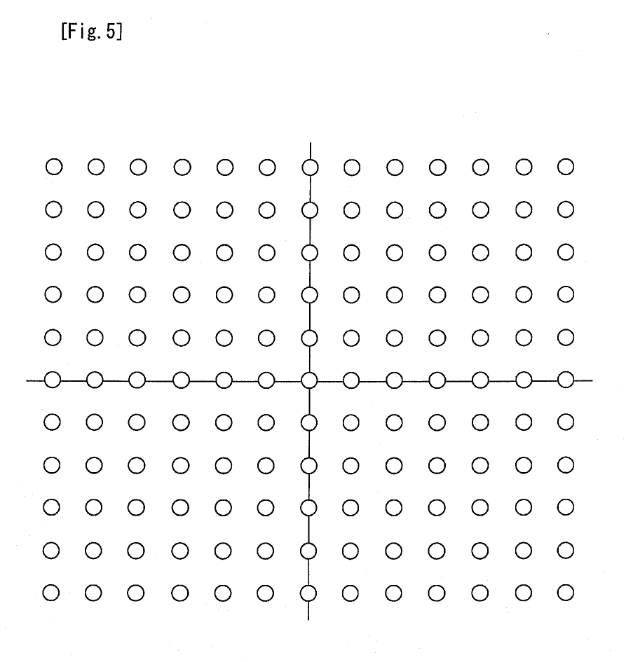

Recognition of an image by vision will be explained. FIG. 4 is an example of a shape to recognize by vision. A black quadrilateral is used for the sake of simplicity. An actual eyeball has a function of sensing not only the brightness of an image but also the color thereof. It is considered that the metamorphopsia mentioned herein has no deep relationship with color, and therefore, the simplified black-and-white image is employed herein. In FIG. 1, the shape of the quadrilateral passes through the cornea 11, is focused through the crystalline lens 13, and is projected on the retina 15. Around the macula 17 of the retina 15, there are many visual cells 19 that are presumed to be randomly arranged. Here, it is assumed that they are regularly arranged in an X-Y plane as illustrated in FIG. 5. Straight lines in the figure are X- and Y-axes on the retina that cross each other at an origin that is assumed to be the center of the macula 17.

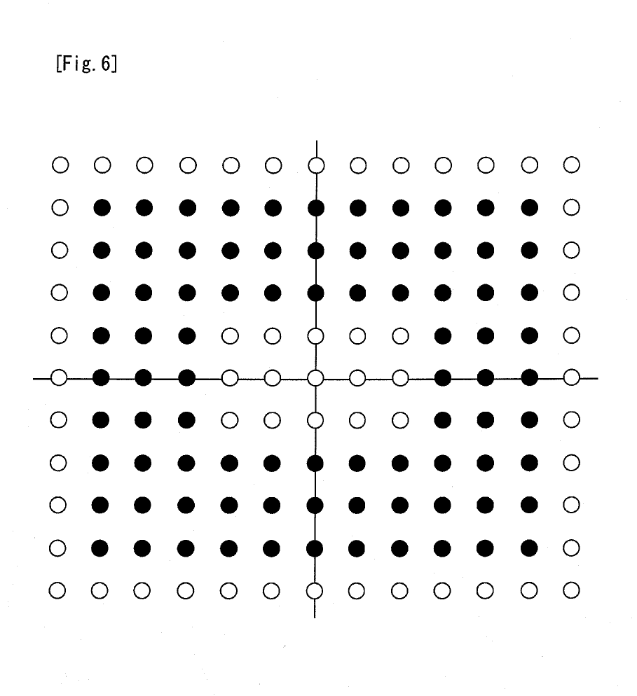

The shape of FIG. 4 projected on the retina 15 is overlaid on the arrangement of visual cells of FIG. 5, to model image information inputted into the visual cells as illustrated in FIG. 6. This is simple binarized information with the black shape illustrated in FIG. 4 being represented with blacks and the white shape in FIG. 4 being represented with whites. The density of visual cells actually present on the retina is further higher than that illustrated in FIG. 5. For the sake of explanation, FIG. 5 assumes that the density of visual cells is low.

The black-and-white information of visual cells illustrated in FIG. 6 is transferred through the optic nerves to the brain. The brain reprocesses the image information according to a preset arrangement of visual cells that is the same as that of FIG. 5 and recognizes the shape illustrated in FIG. 4.

The above is an outline of the function of recognizing an image by vision.

Based on the above-mentioned function, how metamorphopsia occurs when the macula suffers from a disease will be explained.

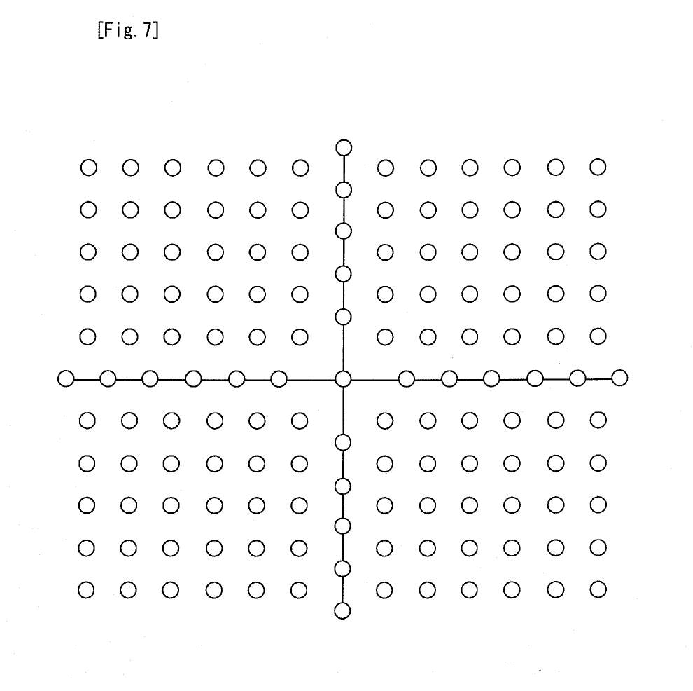

When age-related macular degeneration or the like occurs, a shape around the macular of the retina deforms, and due to the deformation, the arrangement of visual cells on the retina illustrated in FIG. 5 changes. An actual change in the visual cell arrangement is very complicated, and therefore, an assumption is made for the sake of simplicity that the arrangement is distorted as illustrated in FIG. 7. FIG. 7 is similar to FIG. 5 but involves visual cells that have moved in horizontal and vertical directions from original positions on the X- and Y-axes.

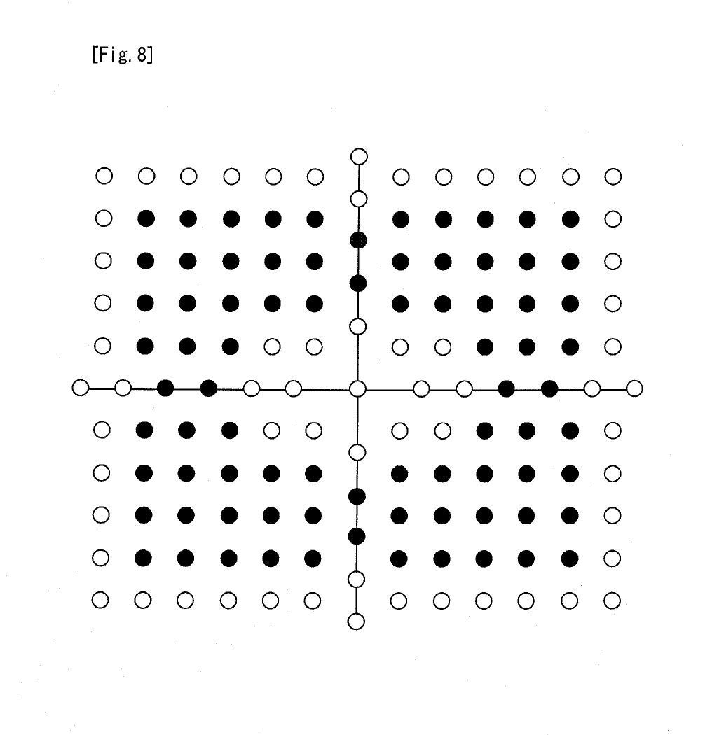

In this state, the black quadrilateral illustrated in FIG. 4 is projected on the retina, to provide visual cell information of FIG. 8. This is obtained like FIG. 6 by overlapping FIG. 4 on FIG. 7. FIG. 8 is similar to FIG. 6 but is slightly different therefrom in information on the X- and Y-axes where the visual cells have moved. Namely, in FIG. 6, visual cells in a plus (right) direction on the X-axis from the center are white, white, black, black, black, and white, i.e., there are three consecutive blacks. In FIG. 8 with positionally changed visual cells, the number of blacks that follow whites is reduced to two from three. This phenomenon also occurs in a minus direction on the X-axis. The same occurs in plus and minus directions on the Y-axis.

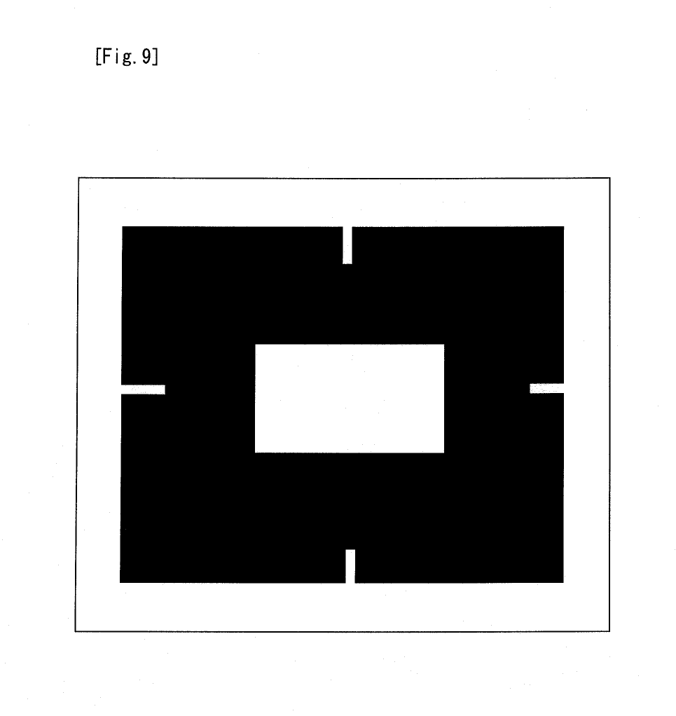

Information about each visual cell thus obtained is sent to the brain similar to the case of FIG. 6. Based on the data sent from the visual cells, the brain reprocesses the image. The brain has no information about the new arrangement of visual cells moved due to the disease such as age-related macular degeneration, and therefore, reprocesses the image information according to the arrangement of visual cells illustrated in FIG. 5. FIG. 9 illustrates the shape recognized by reprocessing the data of FIG. 8. Since the visual cells on the X- and Y-axes have moved as illustrated in FIG. 8, the image illustrated in FIG. 9 is deformed with each end on each of the X- and Y-axes changed from black to white.

For the sake of easy understanding, the above-mentioned example is a simple case in which only the visual cells on the X- and Y-axes move. If the disease actually occurs, many visual cells more than those of FIG. 7 move in a complicated manner, to cause metamorphopsia that deforms an image. A relationship between deformation of the retina and movement of the visual cells will be explained later.

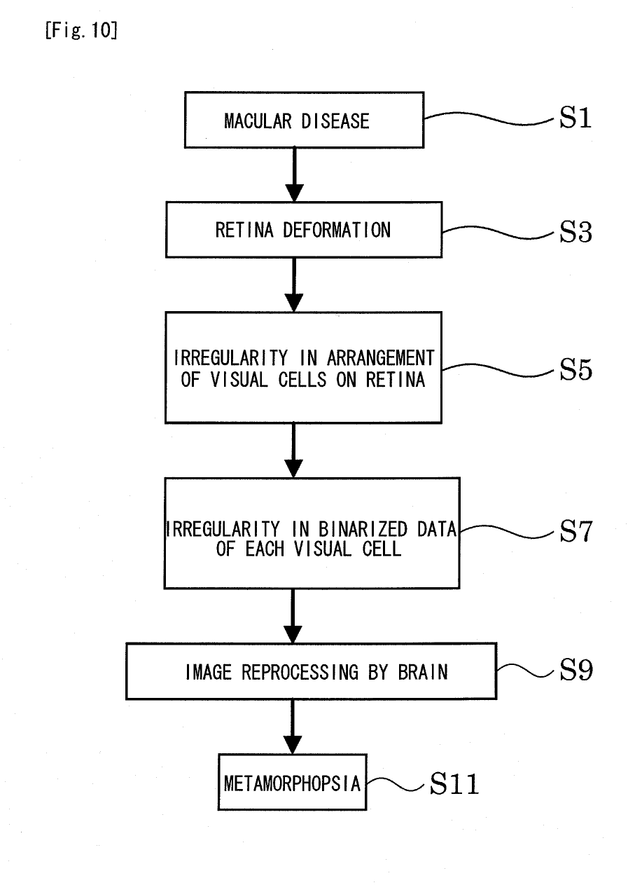

The process of causing metamorphopsia explained above is summarized in a flowchart of FIG. 10. First, the disease occurs in the macula (S1) and the retina deforms (S3). As a result, an arrangement of visual cells on the retina is disordered (S5). When the disordered visual cells capture an image, binarized information on the visual cells becomes disordered (S7). The disordered binarized information is sent to the brain, which reprocesses an image according to a not-disordered arrangement of the visual cells (S9). This causes metamorphopsia to deform an image from its original state (S11).

The metamorphopsia simulator 101 of the present invention is based on the above-mentioned mechanism of causing metamorphopsia, to propose a simulation method and a simulator to quantitatively reproduce an image recognized by a patient who suffers from metamorphopsia.

The flowchart may reversely be followed. Namely, an image of a test chart observed by a metamorphopsia patient is inputted into the input unit 111, a movement amount of visual cells is analyzed according to the image, and a deformation amount of the retina is obtained according to the analysis.

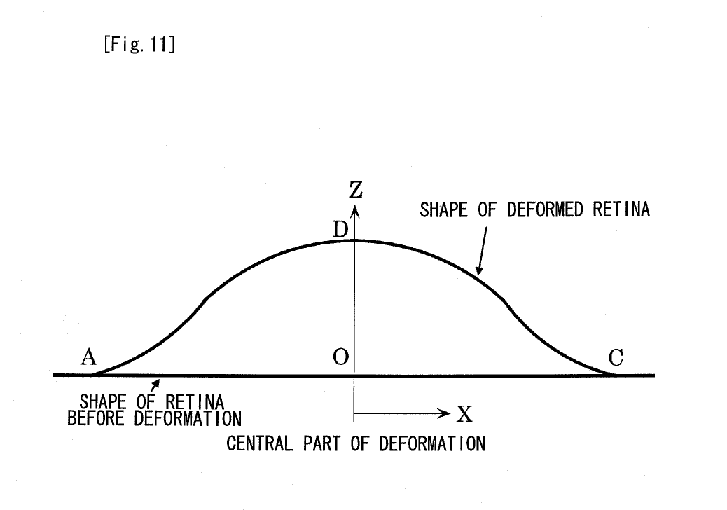

If a disease such as age-related macular degeneration occurs, the retina deforms at the macular illustrated in FIG. 1. FIG. 11 is a sectional view illustrating deformation of the retina around the macular. In the figure, a straight line AOC indicates a surface shape of the retina before deformation. This figure does not consider the curvature of the retina illustrated in FIG. 1 for the sake of simplicity. Even when the curvature is taken into consideration, the same way of thinking is adoptable.

FIG. 11 illustrates, with a curve ADC, a state of the retina deformed due to some disease toward the vitreous body 18, i.e., the inner side. Deformation of the retina occurs three-dimensionally, and therefore, FIG. 11 is turned around the Z-axis, to obtain three-dimensional shapes of the retina surface before and after the deformation. For the sake of simplicity, a deformation center is set on the Z-axis so that the deformation is left-right-symmetrical. Asymmetrical deformation can be handled similarly.

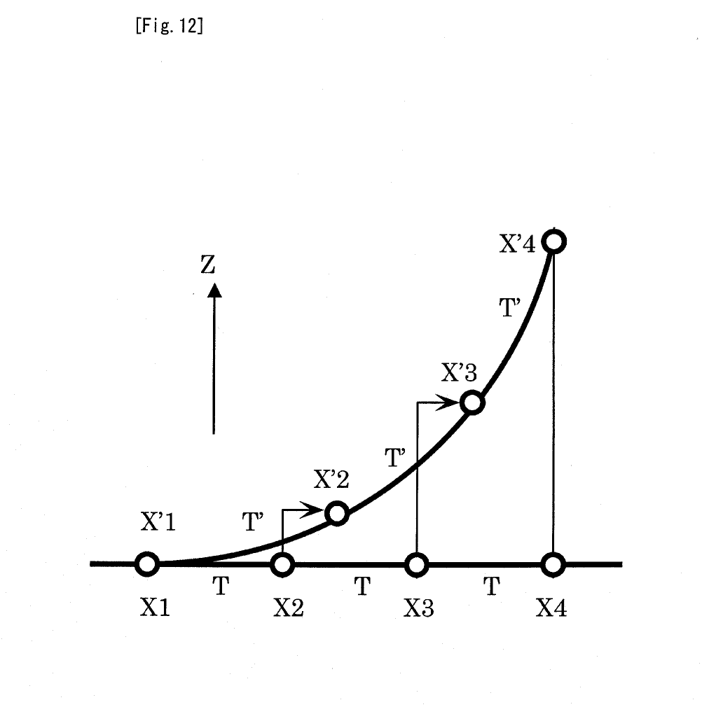

Visual cells are regularly arranged on the retina as illustrated in FIG. 5. Without regard to whether or not the retina deforms, the number of visual cells is fixed. It is understood that a distance between adjoining visual cells is identical to allow the below-mentioned consideration. FIG. 12 illustrates part of the shapes of the retina before and after deformation of FIG. 11. In FIG. 12, visual cells are arranged at regular intervals. It is assumed that there are four visual cells X1, X2, X3, and X4 and a distance between adjoining visual cells is T before deformation. FIG. 12 also shows the retina deforming as illustrated in FIG. 11. In FIG. 12, a left end causes no deformation and a right side bulges, for the sake of simplicity. In this case, the visual cells X1 to X4 upwardly move in the direction of the Z-axis. If the visual cells X1 and X4 do not move in the horizontal direction, the visual cells X2 and X3 move in the right direction. This is because the constant distance T between adjoining visual cells changes after deformation into a constant distance T′.

In this way, the visual cells existing on the retina AOC before deformation move to ADC after deformation not only in the Z-axis direction but also in the horizontal (X-axis) direction. In a three-dimensional arrangement of visual cells turned around the Z-axis, each cell moves according to the principle explained with reference to FIG. 12. Namely, the irregularity in an arrangement of visual cells explained with reference to FIG. 7 entirely occurs over the visual field.

In the metamorphopsia simulator 101 of the embodiment of the present invention, the retina deformation analyzing unit 113 mathematically expresses the shape of the retina illustrated in FIG. 11 and proposes a simulation method based on the principle illustrated in FIGS. 11 and 12.

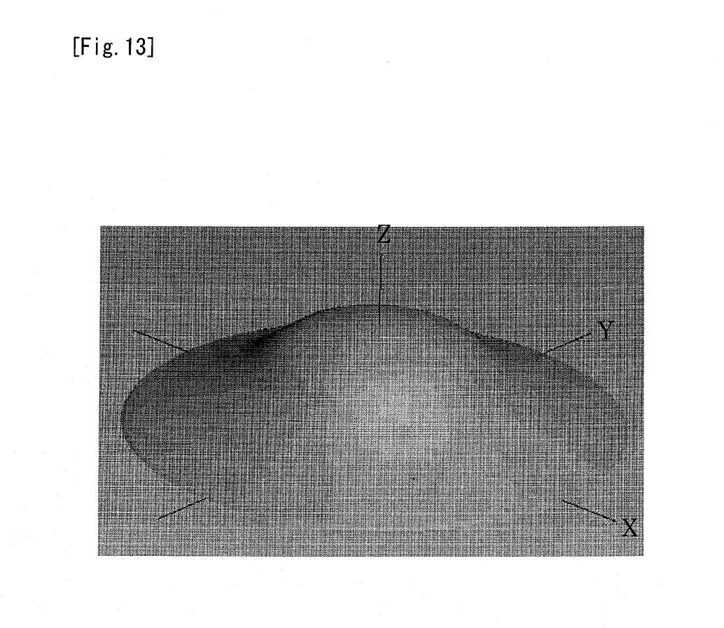

FIG. 13 illustrates a coordinate system used by the present invention. Here, the center of the deformation illustrated in FIG. 11 serves as an origin, and together with the upwardly oriented Z-axis and horizontal X- and Y-axes, provides a three-dimensionally-deformed retina shape. An X-Z section of FIG. 13, i.e., a relationship of the X- and Z-axes after deformation illustrated in FIG. 11 is estimated according to the below-mentioned Expression 1 formed on the basis of a probability density function.

$$Z = A \cdot exp \frac{- {x}^{2}}{2 {σ}^{2}} \qquad ( Expression 1 )$$

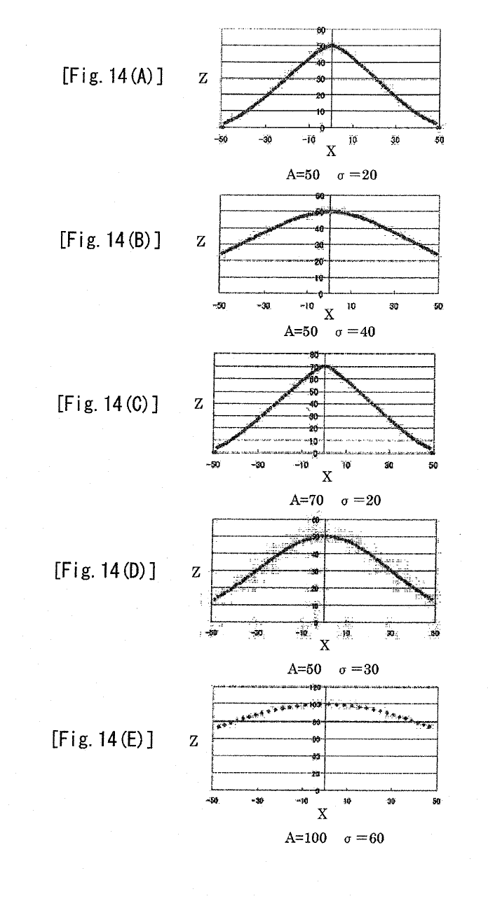

In the Expression 1, a visual line direction is a vertical direction of the retina, a direction orthogonal to the visual line is a horizontal direction of the retina, Z is a movement amount in the vertical direction, and x is a distance from the origin. In this example, the distance takes not a length unit but an integer multiple of the distance T between visual cells illustrated in FIG. 12. “A” is a maximum deformation value of the retina and corresponds to the length of OD in FIG. 11. “σ” indicates a standard deviation according to the probability density function and corresponds to the width of the mountainous shape of FIG. 11. When the deviation is large, the width of the mountain is large to provide a smooth shape, and when it is small, the mountainous shape has a steep peak at the center thereof. FIGS. 14(A)-14(E) examples of sectional shapes of the deformed retina calculated from the Expression 1. Values on ordinate and abscissa are multiple numbers with respect to the distance between visual cells that is set as “1”. From these calculation examples, it is understood that substituting certain values for A and σ of the Expression 1 numerically provides a deformed shape of the retina caused by a macular disease.

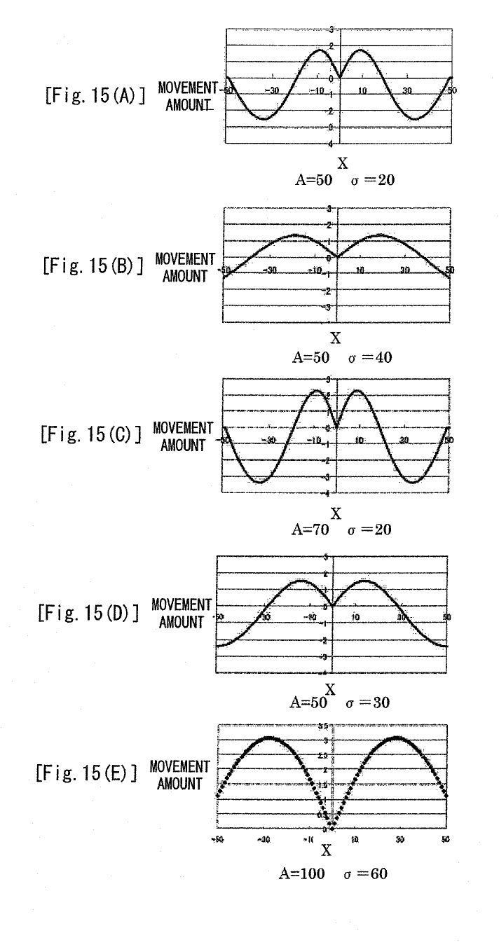

If the retina deforms, visual cells existing on the retina move as illustrated in FIG. 12 not only in the Z-axis direction but also in the horizontal X-axis direction. As illustrated in FIGS. 14(A)-14(E), a movement amount of visual cells is calculable under the condition that the visual cells are present at regular intervals on the deformed retina. Based on this, the visual cell movement analyzing unit 115 calculates, as illustrated in FIGS. 15(A)-15(E), a horizontal movement amount of visual cells existing on the deformed retina illustrated in FIGS. 14(A)-14(E). FIGS. 15(A)-15(E) show calculated horizontal movement amounts of visual cells corresponding to the calculation examples of FIGS. 14(A)-14(E) with a movement of visual cells in a direction parting from the origin, i.e., in a visual cell interval widening direction being plus. For example, in the case of A=50 and σ=20 of FIGS. 15(A)-15(E), a visual cell at the center does not move and stays at the same position before and after deformation, a visual cell just on the outer side of the center cell moves in a direction to widen the interval, and visual cells around the position where the number of the cells is 20 tend to move in a direction to narrow the interval. Movement amounts of visual cells differ depending on the values of A and σ that indicate deformation of the retina.

As illustrated in FIGS. 8 and 9, positional movements of visual cells result in deforming the shape of a perceived image when the image is reprocessed in the brain, thereby causing metamorphopsia.

The metamorphopsic image reproducing unit 117 reproduces distortion of an image observed by the metamorphopsia patient according to the movement amounts of visual cells.

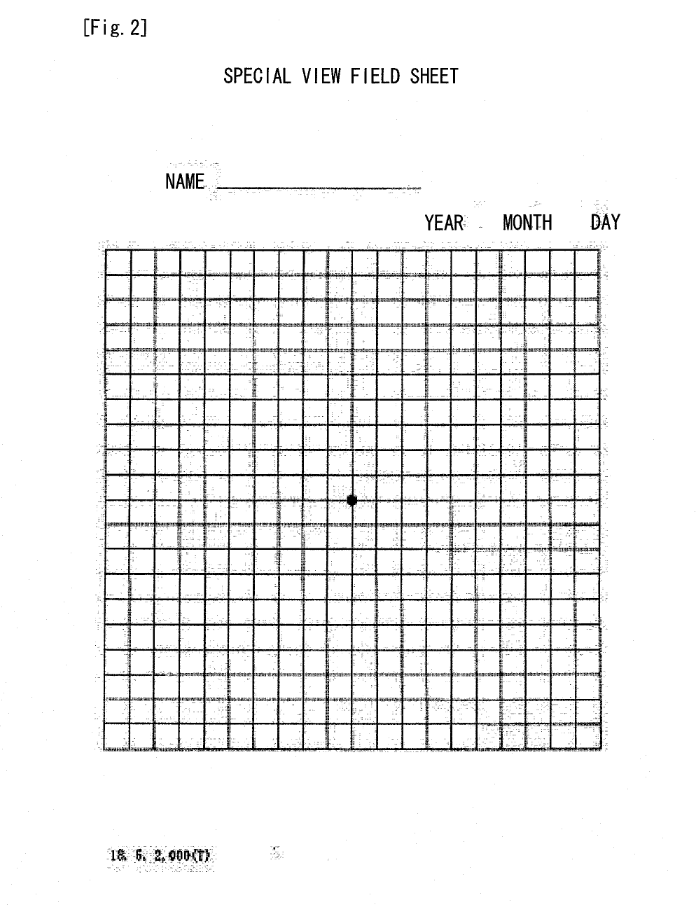

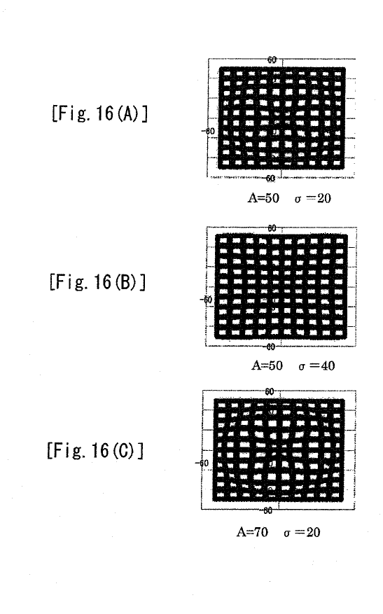

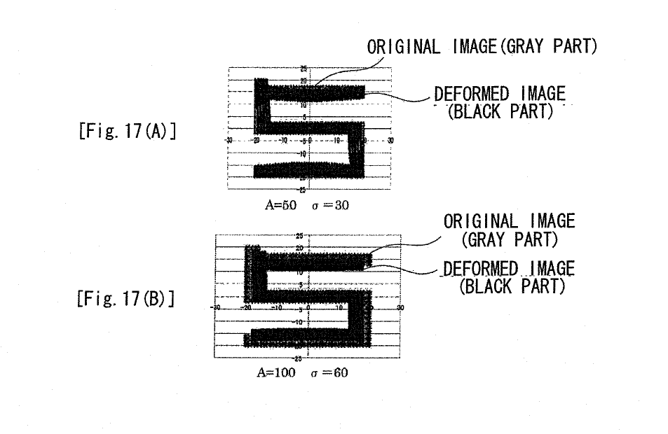

By following the flowchart of FIG. 10, the metamorphopsic image reproducing unit 117 simulates and reproduces an image recognized by the metamorphopsia patient, as illustrated in FIGS. 16(A)-16(C). The examples of FIGS. 16(A)-16(C) are obtained from a vertical-horizontal grid pattern like the Amsler chart of FIG. 2. The examples correspond to the three cases illustrated in FIGS. 14 and 15 ((A), (B), and (C) of FIGS. 14 and 15). It is confirmed that an image deformed by visual cell movement is reproducible. As mentioned herein, the simulator according to the present invention is capable of reproducing an image recognized by a metamorphopsia patient according to the values A and a that indicate a deformation amount of the retina. Based on this technique, the metamorphopsic image reproducing unit 117 is able to simulate distortion of a character as illustrated in FIGS. 17(A) and 17(B). These examples are reproduced under the fourth condition ((D) of FIGS. 14 and 15) and fifth condition ((E) of FIGS. 14 and 15) of FIGS. 14 and 15. In each of the cases ((A) and (B) of FIG. 17), a reproduced image indicated with black is deformed compared with an original image indicated with gray. In each case, the reproduced image is smaller than the original image. This agrees with the testimony of a patient that an image is visible smaller as well as deformation of the image caused by deformation of the macula and verifies the correctness of the simulation proposed by the present invention.

The present invention proposes the method of numerically expressing deformation of the macula caused by a disease and reproducing distortion of an image recognized by a metamorphopsia patient. Although the examples mentioned here relate to left-right symmetric deformation of the macula, or shapes obtained by turning the symmetric deformation around a Z-axis, the simulation is also applicable to asymmetric cases. The deformation examples of the macula illustrated in FIGS. 14(A)-14(E) are obtained from the numerical expression (Expression 1) modified from a probability density function. This modification does not limit the present invention. For example, deformation of the retina around the macula may be divided at predetermined angles into radial shapes spreading from the center thereof toward the periphery and each radial shape may be defined with the expression involving different A and σ. Even for such a case, the simulation technique proposed by the present invention is applicable as it is. As mentioned above, photographing the eyeground enables abnormality in the shape of the macula to be quantitatively measured, and therefore, a result of the measurement is usable for the simulation after digitizing the measurement result.

What the simulator 101 of the embodiment of the present invention realizes will be explained.

The retina deformation analyzing unit 113 analyzes deformation of the retina of a metamorphopsia patient, and based on the analysis, the visual cell movement analyzing unit 115 numerically simulates irregularity in an arrangement of visual cells of the patient.

Based on the numerical simulation, the metamorphopsic image reproducing unit 117 outputs distortion of an image observed by the metamorphopsia patient, so that a doctor or a healthy subject can recognize the distortion of the image observed by the metamorphopsia patient.

The retina deformation analyzing unit 113 approximates a deformation amount of the retina of the metamorphopsia patient according to a numerical expression (Expression 1) that is based on a probability density function. The numerical expression enables a movement amount of visual cells and a metamorphopsic image to be easily analyzed and reproduced and related values to be speedily calculated.

The simulator 101 is able to conduct an opposite simulation. Namely, based on distortion of an image observed by a metamorphopsia patient, it is able to analyze a deviation (movement amount) of visual cells from a normal position, as well as a deformation amount of the retina.

As a result, the simulation according to the present invention is able to obtain a one-to-one relationship between deformation of the macula obtained from an eyeground photograph of the patient and the distorted image recognized by the patient at this time. As a result, a doctor can concretely understand how the patient recognizes the image and can share not only the physical disease but also mental anguish with the patient to care for mental anxiety caused by the physical disease.

Although the embodiment of the present invention has been explained in connection with metamorphopsia among eye diseases, the metamorphopsia simulator 101 according to the embodiment of the present invention is also applicable to the other eye diseases other than metamorphopsia.

1. An ophthalmic simulator, comprising:
a retina deformation analyzing unit mathematically analyzing, based on a shape of a retina after deformation, a deformation amount with respect to a shape of the retina before deformation; and
a visual cell movement analyzing unit mathematically analyzing, based on the deformation amount of the retina analyzed by the retina deformation analyzing unit, a movement amount of visual cells due to the deformation of the retina.

2. The ophthalmic simulator of claim 1, further comprising:
a metamorphopsic image reproducing unit reproducing, based on the movement amount of visual cells analyzed by the visual cell movement analyzing unit, distortion of an image observed by the eye disease patient.

3. The ophthalmic simulator of claim 1, wherein the retina deformation analyzing unit approximates the deformation amount of the retina according to the below-mentioned Expression 1 based on a probability density function with a visual line being in a vertical direction of the retina, a direction orthogonal to the visual line being a horizontal direction of the retina, a movement amount in the vertical direction of the retina being Z, a maximum value of deformation of the retina being A, a distance in the horizontal direction from an origin being x, and a standard deviation in the probability density function being σ:
$$Z = A \cdot exp \frac{- {x}^{2}}{2 {σ}^{2}} . \qquad ( Expression 1 )$$

4. The ophthalmic simulator of claim 2, wherein the retina deformation analyzing unit approximates the deformation amount of the retina according to the below-mentioned Expression 1 based on a probability density function with a visual line being in a vertical direction of the retina, a direction orthogonal to the visual line being a horizontal direction of the retina, a movement amount in the vertical direction of the retina being Z, a maximum value of deformation of the retina being A, a distance in the horizontal direction from an origin being x, and a standard deviation in the probability density function being σ:
$$Z = A \cdot exp \frac{- {x}^{2}}{2 {σ}^{2}} . \qquad ( Expression 1 )$$
